(19) United States
(12) Patent Application Publication
(10) Pub. No.: US 2011/0015155 A1
(43) Pub. Date: Jan. 20, 2011

(54) Deuterated 2, 4-Pyrimidinediamine Compounds and Prodrugs Thereof and Their Uses

(75) Inventors: Somasekhar Bhamidipati, Foster City, CA (US); Rajinder Singh, Belmont, CA (US); Jeffrey Clough, Redwood City, CA (US)

Correspondence Address: McDonnell Boehnen Hulbert & Berghoff LLP, 300 South Wacker Drive, Chicago, IL, 60606, US

(73) Assignee: RIGEL PHARMACEUTICALS, INC., South San Francisco, CA (US)

(21) Appl. No.: 12/838,224

(22) Filed: Jul. 16, 2010

Related U.S. Application Data
(60) Provisional application No. 61/226,551, filed on Jul. 17, 2009.

(51) Int. Cl. A61K 31/675; C07D 498/04; C07F 9/09; A61K 31/5383; A61P 37/08; A61P 17/02; A61P 29/00; A61P 19/02; A61P 37/00; A61P 21/04; A61P 1/04

(52) U.S. Cl. 514/81; 544/105; 568/14; 514/230.5

(57) ABSTRACT

The present disclosure provides biologically active deuterated 2,4-pyrimidinediamine compounds and prodrugs thereof, compositions comprising the deuterated compounds, intermediates and methods for synthesizing the deuterated compounds and methods of using the deuterated compounds in a variety of applications.

### BACKGROUND

1. Field of the Disclosure

The present disclosure relates to biologically active deuterated 2,4-pyrimidinediamine compounds and prodrugs thereof, pharmaceutical compositions comprising the deuterated compounds, intermediates and synthetic methods of making the deuterated compounds and methods of using the deuterated compounds and compositions in a variety of contexts, such as in the treatment or prevention of various diseases.

2. Description of the Related Art

Crosslinking of Fc receptors, such as the high affinity receptor for IgE (FcεRI) and/or the high affinity receptor for IgG (FcγRI) activates a signaling cascade in mast, basophil and other immune cells that results in the release of chemical mediators responsible for numerous adverse events. For example, such crosslinking leads to the release of preformed mediators of Type I (immediate) anaphylactic hypersensitivity reactions, such as histamine, from storage sites in granules via degranulation. It also leads to the synthesis and release of other mediators, including leukotrienes, prostaglandins and platelet-activating factors (PAFs), that play important roles in inflammatory reactions. Additional mediators that are synthesized and released upon crosslinking Fc receptors include cytokines and nitric oxide.

The signaling cascade(s) activated by crosslinking Fc receptors such as FcεRI and/or FcγRI includes an array of cellular proteins. Among the most important intracellular signal propagators are the tyrosine kinases. One important tyrosine kinase involved in the signal transduction pathways associated with crosslinking the FcεRI and/or FcγRI receptors, as well as other signal transduction cascades, is Syk kinase (see Valent et al., 2002, Intl. J. Hematol. 75(4):257-362 for review).

The mediators released as a result of FcεRI and FcγRI receptor cross-linking are responsible for, or play important roles in, the manifestation of numerous adverse events. Recently, various classes of 2,4-pyrimidinediamine compounds have been discovered that inhibit the FcεRI and/or FcγRI signaling cascades, and that have myriad therapeutic uses. See, e.g., U.S. patent application Ser. No. 10/355,543 filed Jan. 31, 2003 (US 2004/0029902A1), international patent application Serial No. PCT/US03/03022 filed Jan. 31, 2003 (WO 03/063794), U.S. patent application Ser. No. 10/631,029 filed Jul. 29, 2003 (US 2007/0060603), international patent application no. PCT/US03/24087 (WO 2004/014382), U.S. patent application Ser. No. 10/903,263 filed Jul. 30, 2004 (US2005/0234049), and international patent application no. PCT/US2004/24716 (WO 2005/016893), each of which is hereby incorporated herein by reference in its entirety. While many of these compounds exhibit good bioavailability properties, in some instances it may be desirable to tailor their solubility or other properties such that their bioavailability via specified routes of administration is optimized.

International patent application no. PCT/US03/03022 filed Jan. 31, 2003 (WO 03/063794), international patent application no. PCT/US07/85313 filed Nov. 20, 2007 (WO 2008/064274), and international patent application no. PCT/US06/01945 filed Jan. 19, 2006 (WO 2006/078846), each of which is hereby incorporated herein by reference in its entirety, disclose a class of 2,4-pyrimidinediamine compounds and prodrugs thereof as being useful in a variety of in vitro and in vivo contexts, including in the treatment and/or prevention of diseases mediated, at least in part, by the activation of Fc receptor signaling cascades. While these compounds are useful in a variety of in vitro and in vivo contexts, there remains a need for compounds with improved effects and increased duration of actions.

### SUMMARY OF THE DISCLOSURE

In a broad aspect, the disclosure provides deuterated 2,4-pyrimidinediamine compounds and prodrugs thereof that have myriad biological activities, and hence therapeutic uses, compositions comprising the deuterated compounds and prodrugs, methods and intermediates useful for synthesizing the deuterated compounds and prodrugs and methods of using the deuterated compounds and prodrugs in a variety of in vitro and in vivo contexts, including in the treatment and/or prevention of diseases mediated, at least in part, by the activation of Fc receptor signaling cascades.

Thus, in one aspect, the invention comprises a compound of formula (I):

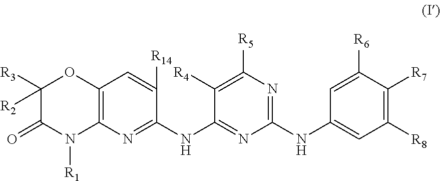

or a pharmaceutically acceptable salt thereof, wherein:
R₁ is selected from hydrogen C₁-C₆ alkyl, and R₉;
R₂ and R₃ are independently selected from hydrogen, halogen, halo(C₁-C₆)alkyl, and C₁-C₆ alkyl, or
R₂ and R₃ together form an oxo group;
R₄ is selected from hydrogen, halogen, cyano, nitro, C₁-C₆ alkyl, and halo(C₁-C₆)alkyl;
R₅ is hydrogen; and
R₆, R₇, and R₈ are independently selected from hydrogen, halogen, cyano, nitro, C₁-C₆ alkyl, C₂-C₆ alkenyl, C₂-C₆ alkynyl, phenyl, benzyl, —OR₁₀, —C(O)R₁₀, —C(O)OR₁₀, —NR₁₀R₁₀, —S(O)₂NR₁₀R₁₀, —C(O)NR₁₀R₁₀, —N(R)S(O)₂R₁₀ and —NC(O)OR₁₀, where each R₁₀ is independently hydrogen or C₁-C₆ alkyl; and
R₉ is —(CR₁₁R₁₁)ₖ—O—P(O)(OR₁₂)₂ or

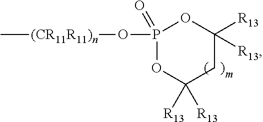

in which k is 1, 2 or 3; m is 0 or 1; n is 1, 2 or 3; each R₁₁ is independently hydrogen or C₁-C₆ alkyl; each R₁₂ is independently hydrogen or C₁-C₆ alkyl, and each R₁₃ is independently hydrogen, or C₁-C₆ alkyl; and
R₁₄ is hydrogen;
provided that at least one hydrogen of the compound is enriched in deuterium.

And in one embodiment of this aspect, the invention comprises compounds of formula (I):

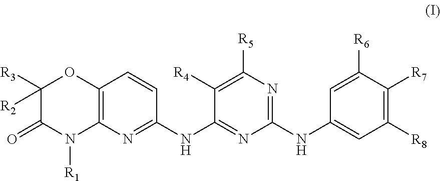

or a pharmaceutically acceptable salt thereof, wherein:
R₁ is selected from hydrogen, deuterium, C₁-C₆ alkyl optionally substituted with deuterium, and R₉;
R₂ and R₃ are independently selected from hydrogen, deuterium, halogen, halo(C₁-C₆)alkyl, and C₁-C₆ alkyl optionally substituted with deuterium, or R₂ and R₃ together form an oxo group;
R₄ is selected from hydrogen, deuterium, halogen, cyano, nitro, C₁-C₆ alkyl, and halo(C₁-C₆)alkyl;
R₅ is hydrogen or deuterium; and
R₆, R₇, and R₈ are independently selected from hydrogen, deuterium, halogen, cyano, nitro, C₁-C₆ alkyl optionally substituted with deuterium, C₂-C₆ alkenyl optionally substituted with deuterium, C₂-C₆ alkynyl optionally substituted with deuterium, phenyl, benzyl, —OR₁₀, —C(O)R₁₀, —C(O)OR₁₀, —NR₁₀R₁₀, —S(O)₂NR₁₀R₁₀, —C(O)NR₁₀R₁₀, —N(R)S(O)₂R₁₀ and —NC(O)OR₁₀,
   where each R₁₀ is independently selected from hydrogen, deuterium, and C₁-C₆ alkyl optionally substituted with deuterium; and
R₉ is selected from —(CR₁₁R₁₁)ₖ—O—P(O)(OR₁₂)₂ and

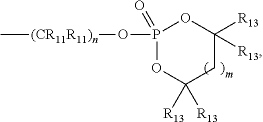

in which k is 1, 2 or 3; m is 0 or 1; n is 1, 2 or 3; each R₁₁ is independently selected from hydrogen, deuterium, and C₁-C₆ alkyl optionally substituted with deuterium; each R₁₂ is independently selected from hydrogen, deuterium, and C₁-C₆ alkyl optionally substituted with deuterium and each R₁₃ is independently selected from hydrogen, deuterium, and C₁-C₆ alkyl optionally substituted with deuterium;

provided that at least one deuterium is present.

Another aspect of the disclosure provides pharmaceutical compositions comprising the compounds of formula (I) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

Another aspect of the disclosure provides a method of inhibiting cell degranulation in a subject, comprising administering to the subject a pharmaceutically effective amount of a compound of formula (I) effective to inhibit degranulation.

Yet another aspect of the disclosure provides a method for treating or preventing a disease selected from an allergic disease, low grade scarring, a disease associated with tissue destruction, a disease associated with tissue inflammation, inflammation and scarring, comprising administering to the subject a pharmaceutically effective amount of a compound of formula (I).

In one aspect, the disclosure provides a method of treating rheumatoid arthritis in a subject, comprising administering to a subject suffering from rheumatoid arthritis a pharmaceutically effective amount of a compound of formula (I).

Another aspect of the disclosure provides a method of inhibiting an activity of a Syk kinase in a subject, comprising administering to the subject a pharmaceutically effective amount of a compound of formula (I) effective to inhibit the Syk kinase activity.

In another aspect, the disclosure provides a method of inhibiting an Fc receptor signal transduction cascade in a subject, comprising administering to the subject a pharmaceutically effective amount of a compound of formula (I) effective to inhibit the Fc receptor signal transduction cascade. Fc receptor is selected from FcαRI, FcγRI, FcγRIII and FcεRI.

Another aspect of the disclosure provides a method of treating or preventing an autoimmune disease in a subject, and/or one or more symptoms associated therewith, comprising administering to the subject a pharmaceutically effective amount of a compound of formula (I) effective to treat or prevent the autoimmune disease.

### DETAILED DESCRIPTION

In one aspect, the invention comprises a compound of formula (I′):

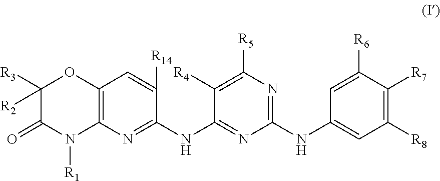

or a pharmaceutically acceptable salt thereof, wherein:
R₁ is selected from hydrogen C₁-C₆ alkyl, and R₉;
R₂ and R₃ are independently selected from hydrogen, halogen, halo(C₁-C₆)alkyl, and C₁-C₆ alkyl, or
R₂ and R₃ together form an oxo group;
R₄ is selected from hydrogen, halogen, cyano, nitro, C₁-C₆ alkyl, and halo(C₁-C₆)alkyl;
R₅ is hydrogen; and
R₆, R₇, and R₈ are independently selected from hydrogen, halogen, cyano, nitro, C₁-C₆ alkyl, C₂-C₆ alkenyl, C₂-C₆ alkynyl, phenyl, benzyl, —OR₁₀, —C(O)R₁₀, —C(O)OR₁₀, —NR₁₀R₁₀, —S(O)₂NR₁₀R₁₀, —C(O)NR₁₀R₁₀, —N(R)S(O)₂R₁₀ and —NC(O)OR₁₀, where each R₁₀ is independently hydrogen or C₁-C₆ alkyl; and
R₉ is —(CR₁₁R₁₁)ₖ—O—P(O)(OR₁₂)₂ or

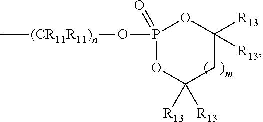

in which k is 1, 2 or 3; m is 0 or 1; n is 1, 2 or 3; each R₁₁ is independently hydrogen or C₁-C₆ alkyl; each R₁₂ is independently hydrogen or C₁-C₆ alkyl, and each R₁₃ is independently hydrogen, or C₁-C₆ alkyl; and
R₁₄ is hydrogen;
provided that at least one hydrogen of the compound is enriched in deuterium.

In one embodiment of this aspect, the invention comprises compounds of formula (I):

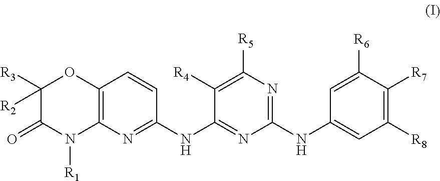

or a pharmaceutically acceptable salt thereof, wherein:
R₁ is selected from hydrogen, deuterium, C₁-C₆ alkyl optionally substituted with deuterium, and R₉;
R₂ and R₃ are independently selected from hydrogen, deuterium, halogen, halo(C₁-C₆)alkyl, and C₁-C₆ alkyl optionally substituted with deuterium, or
R₂ and R₃ together form an oxo group;
R₄ is selected from hydrogen, deuterium, halogen, cyano, nitro, C₁-C₆ alkyl, and halo(C₁-C₆)alkyl;
R₅ is hydrogen or deuterium; and
R₆, R₇, and R₈ are independently selected from hydrogen, deuterium, halogen, cyano, nitro, C₁-C₆ alkyl optionally substituted with deuterium, C₂-C₆ alkenyl optionally substituted with deuterium, C₂-C₆ alkynyl optionally substituted with deuterium, phenyl, benzyl, —OR₁₀, —C(O)R₁₀, —C(O)OR₁₀, —NR₁₀R₁₀, —S(O)₂NR₁₀R₁₀, —C(O)NR₁₀R₁₀, —N(R)S(O)₂R₁₀ and —NC(O)OR₁₀,
   where each R₁₀ is independently selected from hydrogen, deuterium, and C₁-C₆ alkyl optionally substituted with deuterium; and
R₉ is selected from —(CR₁₁R₁₁)ₖ—O—P(O)(OR₁₂)₂ and

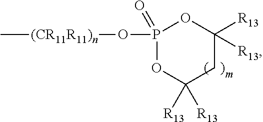

in which k is 1, 2 or 3; m is 0 or 1; n is 1, 2 or 3; each R₁₁ is independently selected from hydrogen, deuterium, and C₁-C₆ alkyl optionally substituted with deuterium; each R₁₂ is independently selected from hydrogen, deuterium, and C₁-C₆ alkyl optionally substituted with deuterium and each R₁₃ is independently selected from hydrogen, deuterium, and C₁-C₆ alkyl optionally substituted with deuterium;

provided that at least one deuterium is present.

In one embodiment, the disclosure provides compounds of formula (I), wherein R₁ is selected from hydrogen, deuterium, and R₉.

In another embodiment, the disclosure provides compounds of formula (I), wherein

R₁ is selected from hydrogen, deuterium, and —(CR₁₁R₁₁)ₖ—O—P(O)(OR₁₂)₂.

In another embodiment, the disclosure provides compounds of formula (I), wherein R₁ is hydrogen or deuterium.

In certain embodiments, the disclosure provides compounds of formula (I), wherein R₁ is —(CR₁₁R₁₁)ₖ—O—P(O)(OR₁₂)₂. In one such embodiment, each R₁₁ is hydrogen. In another such embodiment, each R₁₁ is deuterium.

In certain embodiments, the disclosure provides compounds of formula (I), wherein R₁ is —(CR₁₁R₁₁)—O—P(O)(OH)₂, in which each R₁₁ is independently selected from hydrogen and deuterium.

In one embodiment, the disclosure provides compounds of formula (I), wherein R₁ is —CD₂-O—P(O)(OH)₂.

In another embodiment, the disclosure provides compounds of formula (I) as described above, wherein R₂ and R₃ are independently selected from hydrogen, deuterium, halogen, halo(C₁-C₆)alkyl, and C₁-C₆ alkyl optionally substituted with deuterium.

In yet another embodiment, the disclosure provides compounds of formula (I) as described above, wherein R₂ and R₃ are independently selected from C₁-C₆ alkyl optionally substituted with deuterium.

In one embodiment, the disclosure provides compounds of formula (I), wherein R₂ and R₃ are independently selected from —CH₃, —CH₂D, —CHD₂ and —CD₃.

In one embodiment, the disclosure provides compounds of formula (I) as described above, wherein both of R₂ and R₃ are —CH₃.

In another embodiment, the disclosure provides compounds of formula (I) as described above, wherein both of R₂ and R₃ are —CD₃.

In yet another embodiment, the disclosure provides compounds of formula (I) as described above, wherein R₂ is —CH₃, and R₃ is —CD₃.

In one embodiment, the disclosure provides compounds of formula (I) as described above, wherein R₄ is hydrogen, deuterium, or halogen.

In one embodiment, the disclosure provides compounds of formula (I) as described above, wherein R₄ is halogen. For example, R₄ can be fluorine.

In one embodiment, the disclosure provides compounds of formula (I) as described above, wherein R₅ is hydrogen. In another embodiment, the disclosure provides compounds of formula (I) as described above, wherein R₅ is deuterium.

In one embodiment, the disclosure provides compounds of formula (I) as described above, wherein R₆, R₇, and R₈ are independently selected from —OR₁₀, in which each R₁₀ is independently selected from hydrogen, deuterium, and C₁-C₆ alkyl optionally substituted with deuterium.

In another embodiment, the disclosure provides compounds of formula (I) as described above, wherein R₆, R₇, and R₈ are independently selected from —OR₁₀, in which each R₁₀ is C₁-C₆ alkyl optionally substituted with deuterium.

In yet another embodiment, the disclosure provides compounds of formula (I) as described above, wherein R₆, R₇, and R₈ are independently selected from —OCH₃, —OCH₂D, —OCHD₂ and —OCD₃. In one embodiment, the disclosure provides compounds of formula (I), wherein R₆, R₇ and R₈ are each —OCH₃.

In one embodiment, the disclosure provides compounds of formula (I) as described above, wherein at least one of R₆, R₇, or R₈ is —OCD₃. In certain embodiments, the disclosure provides compounds of formula (I), wherein R₆, R₇ and R₈ are each —OCD₃.

In one embodiment, the disclosure provides compounds of formula (I) as described above, wherein
R₁ is hydrogen or —CH₂—O—P(O)(OH)₂;
R₂ and R₃ are independently C₁-C₆ alkyl;
R₄ is halogen;
R₅ is hydrogen; and
R₆, R₇, and R₈ are independently selected from —OR₁₀, in which each R₁₀ is C₁-C₆ alkyl optionally substituted with deuterium, and
at least one of R₆, R₇, or R₈ contains deuterium.

These compounds can be represented by formula (II)

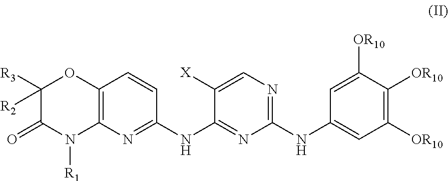

wherein:
R₁ is hydrogen or —CH₂—O—P(O)(OH)₂;
R₂ and R₃ are independently C₁-C₆ alkyl;
X is halogen; and
each R₁₀ is independently C₁-C₆ alkyl optionally substituted with deuterium;
provided that at least one deuterium is present.

In certain embodiments, the disclosure provides compounds of formula (II) as described above, in which R₂ and R₃ are each methyl.

In certain embodiments, the disclosure provides compounds of formula (II) as described above, in which X is fluorine.

In certain embodiments, the disclosure provides compounds of formula (II) as described above, in which at least one R₁₀ is —CD₃, In one embodiment, the disclosure provides compounds of formula (II) as described above, in which all three R₁₀ are —CD₃.

Representative compounds of formula (II) include:

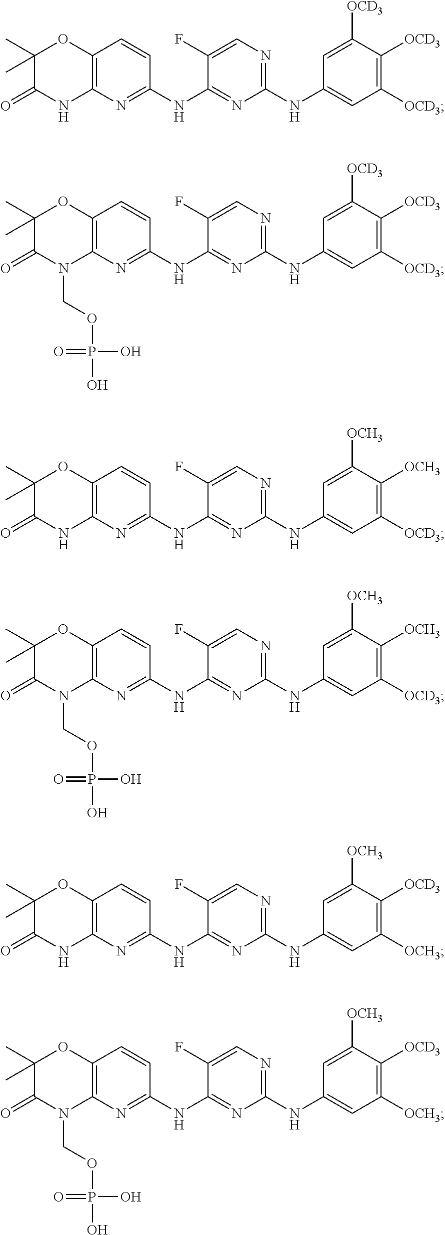

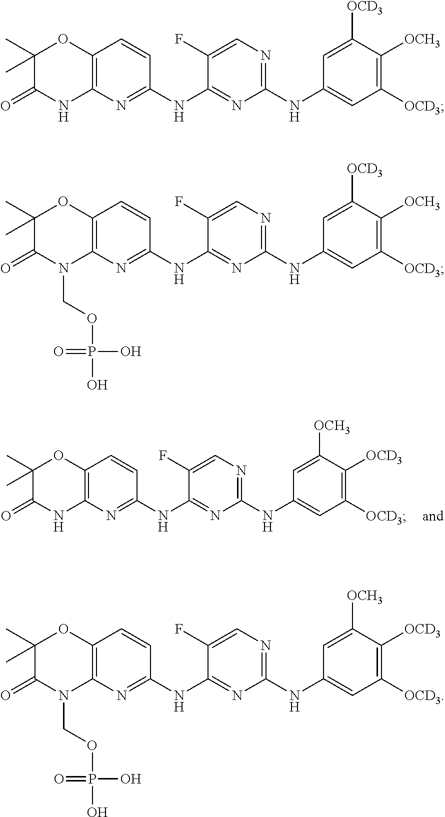

In one embodiment, the disclosure provides compounds of formula (I), wherein R₁ is hydrogen or —CH₂—O—P(O)(OH)₂;
R₂ and R₃ are independently C₁-C₆ alkyl optionally substituted with deuterium, where at least one of R₂ or R₃ contains deuterium;
R₄ is halogen;
R₅ is hydrogen; and
R₆, R₇, and R₈ are independently —O—(C₁-C₆ alkyl).

These compounds can be represented by formula (III)

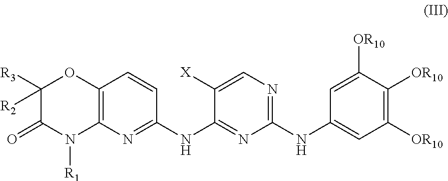

wherein:
R₁ is hydrogen or —CH₂—O—P(O)(OH)₂;
R₂ and R₃ are independently C₁-C₆ alkyl optionally substituted with deuterium, where at least one of R₂ or R₃ contains deuterium;
X is halogen; and
each R₁₀ is independently C₁-C₆ alkyl.

In certain embodiments, the disclosure provides compounds of formula (III) as described above, in which R₂ and R₃ are each —CD₃. In another embodiment, the disclosure provides compounds of formula (III) as described above, in which R₂ is methyl, and R₃ is —CD₃.

In certain embodiments, the disclosure provides compounds of formula (III) as described above, in which X is fluorine.

In certain embodiments, the disclosure provides compounds of formula (III) as described above, in which all three R₁₀ are methyl.

Representative compounds of formula (III) include:

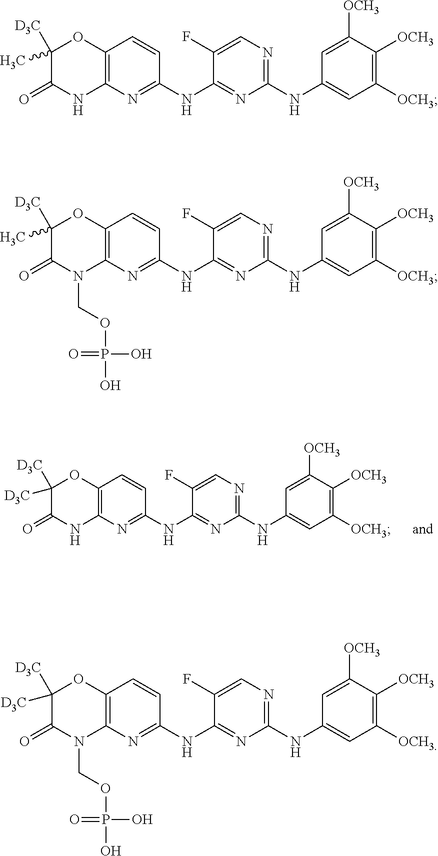

In one embodiment, the disclosure provides compounds of formula (I), wherein
R₁ is —CD₂-O—P(O)(OH)₂;
R₂ and R₃ are independently C₁-C₆ alkyl;
R₄ is halogen;
R₅ is hydrogen; and
R₆, R₇, and R₈ are independently selected from —O—(C₁-C₆ alkyl).

These compounds can be represented by formula (IV)

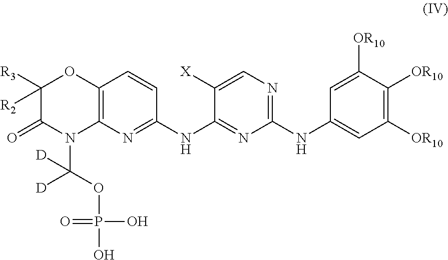

wherein:
R₂ and R₃ are independently C₁-C₆ alkyl;
X is halogen; and
each R₁₀ is independently C₁-C₆ alkyl.

In certain embodiments, the disclosure provides compounds of formula (IV) as described above, in which R₂ and R₃ are each —CH₃.

In certain embodiments, the disclosure provides compounds of formula (IV) as described above, in which X is fluorine.

In certain embodiments, the disclosure provides compounds of formula (IV) as described above, in which all three R₁₀ are methyl.

One representative compound of formula (IV) is:

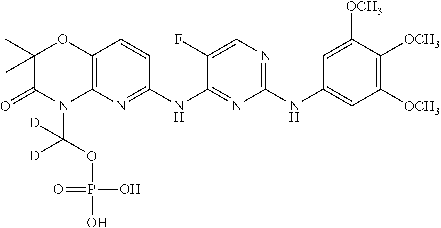

In one embodiment, the disclosure provides compounds of formula (I), wherein
R₁ is hydrogen or —CH₂—O—P(O)(OH)₂;
R₂ and R₃ are independently C₁-C₆ alkyl;
R₄ is halogen;
R₅ is deuterium; and
R₆, R₇, and R₈ are independently selected from —O—(C₁-C₆ alkyl).

These compounds can be represented by formula (V)

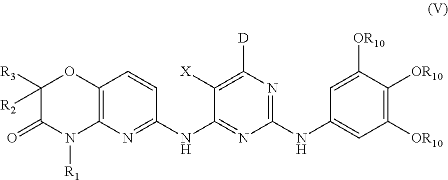

wherein:
R₁ is hydrogen or —CH₂—O—P(O)(OH)₂;
R₂ and R₃ are independently C₁-C₆ alkyl;
X is halogen; and
each R₁₀ is independently C₁-C₆ alkyl.

In certain embodiments, the disclosure provides compounds of formula (V) as described above, in which R₂ and R₃ are each —CH₃.

In certain embodiments, the disclosure provides compounds of formula (V) as described above, in which X is fluorine.

In certain embodiments, the disclosure provides compounds of formula (V) as described above, in which all three R₁₀ are methyl.

Representative compounds of formula (V) include:

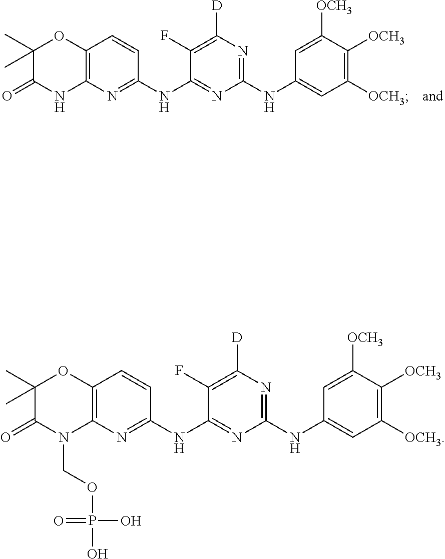

In one embodiment, the disclosure provides compounds of formula (I) as described above, wherein at least two of R₁, R₂, R₃, R₅, R₆, R₇, and R₈ contain deuterium.

In one embodiment, the disclosure provides compounds of formula (I) as described above, wherein at least three of R₁, R₂, R₃, R₅, R₆, R₇, and R₈ contain deuterium.

In one embodiment, the compounds of formula (I) are substituted with progroup R₉ that metabolizes or otherwise transforms under conditions of use to yield the active 2,4-pyrimidinediamine compounds. The progroups R₉ include phosphate moieties that can be cleaved in vitro by enzymes such as esterases, lipases and/or phosphatases. Such enzymes are prevalent throughout the body, residing in, for example, the stomach and digestive tract, blood and/or serum, and in virtually all tissues and organs. Such phosphate-containing R₉ will generally increase the water-solubility of the underlying active 2,4-pyrimidinediamine compound, making such phosphate-containing compounds suitable for modes of administration where water-solubility is desirable, such as, for example, oral, buccal, intravenous, intramuscular and ocular modes of administration.

In one embodiment, R₉ is of the formula —(CR₁₁R₁₁)ₖ—O—P(O)(OH)₂, or a salt thereof, wherein R₁₁ is as previously defined and k is 1 or 2. In certain embodiments, the R₉ group is —CH₂—O—P(O)(OH)₂ or —CD₂-O—P(O)(OH)₂, or a salt thereof. In other embodiments, the R₉ group is —CH₂CH₂—O—P(O)(OH)₂, —CH₂CD₂-O—P(O)(OH)₂, or —CD₂CH₂—O—P(O)(OH)₂, —CD₂CD₂-O—P(O)(OH)₂, or a salt thereof. The salts can be, for example, mono- or di-sodium salts, mono- or di-potassium salts, mono- or di-lithium salts, calcium salts, magnesium salts or ammonium salts.

In another embodiment, R₉ is a cyclic phosphate ester of the formula

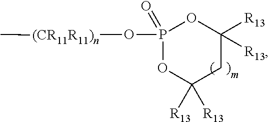

where R₁₁ and R₁₃ are as previously defined, m is 0 or 1, and n is 1 or 2. In certain embodiments, each R₁₁ is hydrogen. In other embodiments, each R₁₁ is deuterium. Specific examples of such cyclic phosphate esters include, but are not limited to, groups selected from:

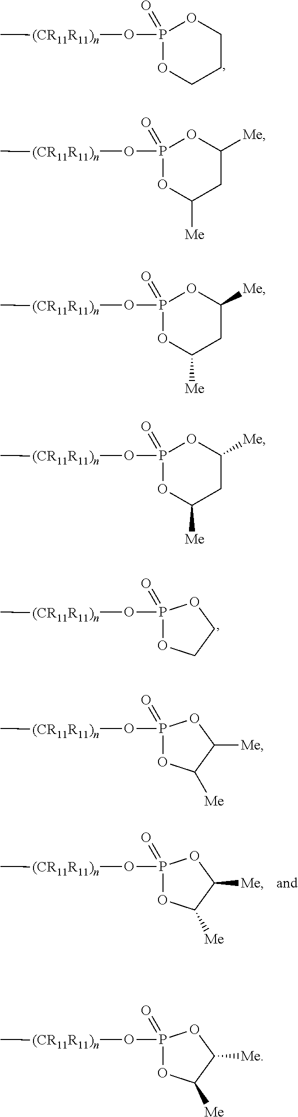

In such examples, the —(CR₁₁R₁₁)ₙ— moiety can, in certain embodiments, be —CD₂- or —CH₂—.

In another aspect, the disclosure provides di-tent-butyl chlorodideuteromethyl phosphate, which has the following structure:

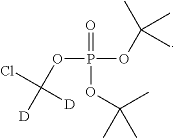

Di-tert-butyl chlorodideuteromethyl phosphate is useful for preparing the 2,4-pyrimidinediamine prodrugs of the disclosure.

In another aspect of the disclosure, di-tent-butyl chlorodideuteromethyl phosphate is prepared from a di-tent-butyl phosphate salt. In one embodiment, for example, the di-tert-butyl phosphate salt is silver di-tert-butylphosphate or tetrabutylammonium di-tert-butylphosphate. The di-tent-butyl phosphate salt can, for example, be reacted with CD₂ICl in the presence of solvent to yield di-tent-butyl chlorodideuteromethyl phosphate.

In another aspect, the disclosure provides a method of preparation of the compound having the formula:

X₁—(CR₁₁R₁₁)ₖ—O—P(O)(OH)₂

wherein k is 1, 2 or 3; X₁ is halogen; and each R₁₁ is independently selected from hydrogen, deuterium, and C₁-C₆ alkyl optionally substituted with deuterium, the method comprising reacting a di-tent-butyl phosphate salt with X₁—(CR₁₁R₁₁)ₖ—X₂, in which X₂ is halogen and can be the same as or different from X₁. For example, in certain embodiments, X₁ is different from X₂. In one embodiment, X₁ is Cl and X₂ is I. Suitable di-tert-butylphosphate salts include, for example, silver di-tent-butyl phosphate and tetrabutylammonium di-tent-butyl phosphate. In certain embodiments, k is 1 or 2. For example, k can be 1.

In one embodiment of the disclosure, incorporation of a heavy atom, particularly substitution of hydrogen with deuterium, into the compounds of formula (I) can give rise to an isotope effect that can alter the pharmacokinetics of the compound. Stable isotope labeling of the compound of the disclosure can alter its physicochemical properties such as pKa and lipid solubility. These changes may influence the fate of the compound at different steps along its passage through the body. Absorption, distribution, metabolism or excretion can be changed. The deuterated compound can have an increased effect and an increased duration of action on mammals at lower concentration than the undeuterated compound.

Deuterium has a natural abundance of about 0.015%. Accordingly, for approximately every 6,500 hydrogen atoms occurring in nature, there is one deuterium atom. Disclosed herein are compounds enriched in deuterium at one or more positions. Thus, deuterium containing compounds of the disclosure have deuterium at one or more positions (as the case may be) in an abundance of greater than 0.015%. So, as used herein, when a moiety or compound is described as “substituted with deuterium” or when a moiety or compound is described as having a deuterium present, it is meant that there is an enrichment in deuterium at the position at which the deuterium is substituted or present.

In one embodiment, a compound of formula (I), at a position designated as having deuterium, has a minimum isotopic enrichment factor of at least 2000 (30% deuterium incorporation) at each atom designated as deuterium in the compound, or at least 3000 (45% deuterium incorporation).

In other embodiments, a compound of formula (I) has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In another embodiment, the disclosure provides pharmaceutically acceptable salts of compounds of formula (I). Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In one embodiment, the disclosure provides the pharmaceutically acceptable salts of compounds of formula (I), wherein R₁ is —(CR₁₁R₁₁)ₖ—O—P(O)(OR₁₂)₂ in which at least one R₁₂ is hydrogen or deuterium.

Pharmaceutically acceptable salts also include salts formed when an acidic proton present in the parent compound is either replaced by an inorganic ion (e.g., an alkali metal ion such as Na⁺, K⁺ or Li⁺, an alkaline earth ion such as Ca²⁺ or Mg²⁺, an aluminum ion, or an ammonium ion) or coordinates with an organic base (e.g., ethanolamine, diethanolamine, triethanolamine, N-methylglucamine, morpholine, piperidine, dimethylamine, diethylamine, etc.).

Specific exemplary salts include, but are not limited to, mono- and di-sodium salts, mono- and di-potassium salts, mono- and di-lithium salts, mono- and di-alkylamino salts, mono- and di-ammonium salts, mono-magnesium salts, and mono-calcium salts.

The compounds described herein, as well as the salts thereof, may also be in the form of hydrates, solvates and N-oxides, as are well-known in the art.

In another aspect, the present disclosure provides compositions comprising one or more of the compounds described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

Many of the compounds described herein, and in particular the compounds according to structural formula (I), are potent inhibitors of degranulation of immune cells, such as mast, basophil, neutrophil and/or eosinophil cells or metabolize to yield 2,4-pyrimidinediamine compounds that potent inhibitors of degranulation of immune cells, such as mast, basophil, neutrophil and/or eosinophil cells. Thus, in still another aspect, the present disclosure provides methods of regulating, and in particular inhibiting, degranulation of such cells. The method generally involves contacting a cell that degranulates with an amount of a suitable compound described herein, or an acceptable salt thereof, effective to regulate or inhibit degranulation of the cell. The method may be practiced in in vitro contexts or in in vivo contexts as a therapeutic approach towards the treatment or prevention of diseases characterized by, caused by or associated with cellular degranulation.

While not intending to be bound by any theory of operation, biochemical data confirm that many 2,4-pyrimidinediamine compounds exert their degranulation inhibitory effect, at least in part, by blocking or inhibiting the signal transduction cascade(s) initiated by crosslinking of the high affinity Fc receptors for IgE (“FcεRI”) and/or IgG (“FcγRI”) (see, e.g., U.S. patent application Ser. No. 10/631,029 filed Jul. 29, 2003 (US 2007/0060603), international patent application no. PCT/US03/24087 (WO2004/014382), U.S. patent application Ser. No. 10/903,263 filed Jul. 30, 2004 (US2005/0234049), and international patent application no. PCT/US2004/24716 (WO 2005/016893), the disclosures of which are hereby incorporated herein by reference.) Indeed, these active 2,4-pyrimidinediamine compounds are potent inhibitors of both FcεRI-mediated and FcγRI-mediated degranulation. As a consequence, the compounds described herein may be used to inhibit these Fc receptor signaling cascades in any cell type expressing such FcεRI and/or FcγRI receptors including but not limited to macrophages, mast, basophil, neutrophil and/or eosinophil cells.

The methods also permit the regulation of, and in particular the inhibition of, downstream processes that result as a consequence of activating such Fc receptor signaling cascade(s). Such downstream processes include, but are not limited to, FcεRI-mediated and/or FcγRI-mediated degranulation, cytokine production and/or the production and/or release of lipid mediators such as leukotrienes and prostaglandins. The method generally involves contacting a cell expressing an Fc receptor, such as one of the cell types discussed above, with an amount of a compound described herein, or an acceptable salt thereof, effective to regulate or inhibit the Fc receptor signaling cascade and/or a downstream process effected by the activation of this signaling cascade. The method may be practiced in in vitro contexts or in in vivo contexts as a therapeutic approach towards the treatment or prevention of diseases characterized by, caused by or associated with the Fc receptor signaling cascade, such as diseases effected by the release of granule specific chemical mediators upon degranulation, the release and/or synthesis of cytokines and/or the release and/or synthesis of lipid mediators such as leukotrienes and prostaglandins.

In yet another aspect, the present disclosure provides methods of treating and/or preventing diseases characterized by, caused by or associated with the release of chemical mediators as a consequence of activating Fc receptor signaling cascades, such as FcεRI and/or FcγRI-signaling cascades. The methods may be practiced in animals in veterinary contexts or in humans. The methods generally involve administering to an animal subject or a human an amount of a compound described herein, or an acceptable salt thereof, effective to treat or prevent the disease. As discussed previously, activation of the FcεRI or FcγRI receptor signaling cascade in certain immune cells leads to the release and/or synthesis of a variety of chemical substances that are pharmacological mediators of a wide variety of diseases. Any of these diseases may be treated or prevented according to the methods described herein.

For example, in mast cells and basophil cells, activation of the FcεRI or FcγRI signaling cascade leads to the immediate (i.e., within 1-3 min. of receptor activation) release of preformed mediators of atopic and/or Type I hypersensitivity reactions (e.g., histamine, proteases such as tryptase, etc.) via the degranulation process. Such atopic or Type I hypersensitivity reactions include, but are not limited to, anaphylactic reactions to environmental and other allergens (e.g., pollens, insect and/or animal venoms, foods, drugs, contrast dyes, etc.), anaphylactoid reactions, hay fever, allergic conjunctivitis, allergic rhinitis, allergic asthma, atopic dermatitis, eczema, urticaria, mucosal disorders, tissue disorders and certain gastrointestinal disorders.

The immediate release of the preformed mediators via degranulation is followed by the release and/or synthesis of a variety of other chemical mediators, including, among other things, platelet activating factor (PAF), prostaglandins and leukotrienes (e.g., LTC4) and the de novo synthesis and release of cytokines such as TNFα, IL-4, IL-5, IL-6, IL-13, etc. The first of these two processes occurs approximately 3-30 min. following receptor activation; the latter approximately 30 min.-7 hrs. following receptor activation. These “late stage” mediators are thought to be in part responsible for the chronic symptoms of the above-listed atopic and Type I hypersensitivity reactions, and in addition are chemical mediators of inflammation and inflammatory diseases (e.g., osteoarthritis, inflammatory bowel disease, ulcerative colitis, Crohn's disease, idiopathic inflammatory bowel disease, irritable bowel syndrome, spastic colon, etc.), low grade scarring (e.g., scleroderma, increased fibrosis, keloids, post-surgical scars, pulmonary fibrosis, vascular spasms, migraine, reperfusion injury and post myocardial infarction), and sicca complex or syndrome. All of these diseases may be treated or prevented according to the methods described herein.

Additional diseases that can be treated or prevented according to the methods described herein include diseases associated with basophil cell and/or mast cell pathology. Examples of such diseases include, but are not limited to, diseases of the skin such as scleroderma, cardiac diseases such as post myocardial infarction, pulmonary diseases such as pulmonary muscle changes or remodeling and chronic obstructive pulmonary disease (COPD), diseases of the gut such as inflammatory bowel syndrome (spastic colon), acute mycloid leukemia (AML) and immune thrombocytopenic purpura.

Many of the active 2,4-pyrimidinediamine compounds are also potent inhibitors of the tyrosine kinase Syk kinase. Examples of such 2,4-pyrimidinediamine are described in the applications referenced above. Thus, in still another aspect, the present disclosure provides methods of regulating, and in particular inhibiting, Syk kinase activity. The method generally involves contacting a Syk kinase or a cell comprising a Syk kinase with an amount of a suitable compound, or an acceptable salt thereof, effective to regulate or inhibit Syk kinase activity. In one embodiment, the Syk kinase is an isolated or recombinant Syk kinase. In another embodiment, the Syk kinase is an endogenous or recombinant Syk kinase expressed by a cell, for example a mast cell or a basophil cell. The method may be practiced in in vitro contexts or in in vivo contexts as a therapeutic approach towards the treatment or prevention of diseases characterized by, caused by or associated with Syk kinase activity.

While not intending to be bound by any particular theory of operation, it is believed that such active 2,4-pyrimdinediamine compounds inhibit cellular degranulation and/or the release of other chemical mediators primarily by inhibiting Syk kinase that gets activated through the gamma chain homodimer of FcεRI. This gamma chain homodimer is shared by other Fc receptors, including FcγRI, FcγRIII and FcαRI. For all of these receptors, intracellular signal transduction is mediated by the common gamma chain homodimer. Binding and aggregation of those receptors results in the recruitment and activation of tyrosine kinases such as Syk kinase. As a consequence of these common signaling activities, the compounds described herein may be used to regulate, and in particular inhibit, the signaling cascades of Fc receptors having this gamma chain homodimer, such as FcεRI, FcγRI, FcγRIII and FcαRI, as well as the cellular responses elicited through these receptors.

Syk kinase is known to play a critical role in other signaling cascades. For example, Syk kinase is an effector of B-cell receptor (BCR) signaling and is an essential component of integrin beta(1), beta(2) and beta(3) signaling in neutrophils. Active 2,4-pyrimidinediamine compounds that are potent inhibitors of Syk kinase can be used to regulate, and in particular inhibit, any signaling cascade where Syk plays a role, such as, fore example, the Fc receptor, BCR and integrin signaling cascades, as well as the cellular responses elicited through these signaling cascades. Thus, the compounds described herein can be used to regulate such activities. The particular cellular response regulated or inhibited will depend, in part, on the specific cell type and receptor signaling cascade, as is well known in the art. Non-limiting examples of cellular responses that may be regulated or inhibited with such compounds include a respiratory burst, cellular adhesion, cellular degranulation, cell spreading, cell migration, phagocytosis (e.g., in macrophages), calcium ion flux (e.g., in mast, basophil, neutrophil, eosinophil and B-cells), platelet aggregation, and cell maturation (e.g., in B-cells).

Thus, in another aspect, the present disclosure provides methods of regulating, and in particular inhibiting, signal transduction cascades in which Syk plays a role. The method generally involves contacting a Syk-dependent receptor or a cell expressing a Syk-dependent receptor with an amount of a suitable compound described herein, or an acceptable salt thereof, effective to regulate or inhibit the signal transduction cascade. The methods may also be used to regulate, and in particular inhibit, downstream processes or cellular responses elicited by activation of the particular Syk-dependent signal transduction cascade. The methods may be practiced to regulate any signal transduction cascade where Syk is now known or later discovered to play a role. The methods may be practiced in in vitro contexts or in in vivo contexts as a therapeutic approach towards the treatment or prevention of diseases characterized by, caused by or associated with activation of the Syk-dependent signal transduction cascade. Non-limited examples of such diseases include those previously discussed.

Recent studies have shown that activation of platelets by collagen is mediated through the same pathway used by immune receptors, with an immunoreceptor tyronsine kinase motif on the FcRγ playing a pivotal role, and also that FcRγ plays a pivotal role in the generation of neointimal hyperplasia following balloon injury in mice, most likely through collagen-induced activation of platelets and leukocyte recruitment. Thus, the compounds described herein can also be used to inhibit collagen-induced platelet activation and to treat or prevent diseases associated with or caused by such platelet activation, such as, for example, intimal hyperplasia and restenosis following vascular injury.

Cellular and animal data also confirm that many of these active 2,4-pyrimidinediamine compounds may also be used to treat or prevent autoimmune diseases and/or symptoms of such diseases. As a consequence, active 2,4-pyrimidinediamine compounds can likewise be used to treat or prevent such autoimmune diseases and/or symptoms. The methods generally involve administering to a subject suffering from an autoimmune disease or at risk of developing an autoimmune disease an amount of a suitable compound described herein, or an acceptable salt thereof, effective to treat or prevent the autoimmune disease and/or its associated symptoms. Autoimmune diseases that can be treated or prevented with the compounds described herein include those diseases that are commonly associated with nonanaphylactic hypersensitivity reactions (Type II, Type III and/or Type IV hypersensitivity reactions) and/or those diseases that are mediated, at least in part, by activation of the FcγR signaling cascade in monocyte cells. Such autoimmune disease include, but are not limited to, those autoimmune diseases that are frequently designated as single organ or single cell-type autoimmune disorders and those autoimmune disease that are frequently designated as involving systemic autoimmune disorder. Non-limiting examples of diseases frequently designated as single organ or single cell-type autoimmune disorders include: Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, chronic aggressive hepatitis, ulcerative colitis and membranous glomerulopathy. Non-limiting examples of diseases often designated as involving systemic autoimmune disorder include: systemic lupus erythematosis, rheumatoid arthritis, Sjogren's syndrome, Reiter's syndrome, polymyositis-dermatomyositis, systemic sclerosis, polyarteritis nodosa, multiple sclerosis and bullous pemphigoid. Additional autoimmune diseases, which can be β-cell (humoral) based or T-cell based, include autoimmune alopecia, Type I or juvenile onset diabetes, and thyroiditis.

### DEFINITIONS

As used herein, the following terms are intended to have the following meanings:

“Alkyl” includes those alkyl groups of a designated number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like.

The term “alkenyl” as used herein, means a straight or branched chain hydrocarbon containing from 2 to 10, preferably 2 to 6, carbons, and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkynyl” as used herein, means a straight or branched chain hydrocarbon group containing from 2 to 10, preferably 2 to 6, carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The terms “halogen” or “halo” indicate fluorine, chlorine, bromine, and iodine.

The term “haloalkyl” refers to an alkyl group substituted with one or more halogen atoms, where each halogen is independently F, Cl, Br or I. Preferred halogens are F and Cl. Preferred haloalkyl groups contain 1-6 carbons, more preferably 1-4 carbons, and still more preferably 1-2 carbons. “Haloalkyl” includes perhaloalkyl groups, such as CF₃ or CF₂CF₃. A preferred haloalkyl group is trifluoromethyl. “Halo(C₁-C₆)alkyl” denotes a haloalkyl having in the range of from 1 to 6 carbons.

The term “isotopic enrichment factor” as used herein means the ratio between the isotopic abundance and the natural abundance of a specified isotope. It will be recognized that some variation of natural isotopic abundance occurs in a synthesized compound depending upon the origin of chemical materials used in the synthesis. Thus, a preparation of any compound will inherently contain small amounts of deuterated isotopologues. The concentration of naturally abundant stable hydrogen isotopes, notwithstanding this variation, is small and immaterial as compared to the degree of stable isotopic substitution of compounds of this disclosure. In a compound of this disclosure, when a particular position is designated as having deuterium, it is understood that the abundance of deuterium at that position is substantially greater than the natural abundance of deuterium, which is about 0.015% (on a mol/mol basis). A position designated as having deuterium will often have a minimum isotopic enrichment factor of at least 3000 (45% deuterium incorporation) at each atom designated as deuterium in the compound.

In the compounds of this disclosure any atom not specifically designated as a particular isotope is meant to represent any stable isotope of that atom. Unless otherwise stated, when a position is designated specifically as “H” or “hydrogen”, the position is understood to have hydrogen at about its natural abundance isotopic composition.

The term “isotopologue” refers to a species that has the same chemical structure and formula as a specific compound of this disclosure, with the exception of the isotopic composition at one or more positions, e.g., H vs. D. Thus an isotopologue differs from a specific compound of this disclosure in the isotopic composition thereof.

“Fc Receptor” refers to a member of the family of cell surface molecules that binds the Fc portion (containing the specific constant region) of an immunoglobulin. Each Fc receptor binds immunoglobulins of a specific type. For example the Fcα receptor (“FcαR”) binds IgA, the FcεR binds IgE and the FcγR binds IgG.

The FcαR family includes the polymeric Ig receptor involved in epithelial transport of IgA/IgM, the myeloid specific receptor RcαRI (also called CD89), the Fcα/μR and at least two alternative IgA receptors (for a recent review see Monteiro & van de Winkel, 2003, Annu Rev. Immunol, advanced e-publication). The FcαRI is expressed on neutrophils, eosinophils, monocytes/macrophages, dendritic cells and kupfer cells. The FcαRI includes one alpha chain and the FcR gamma homodimer that bears an activation motif (ITAM) in the cytoplasmic domain and phosphorylates Syk kinase.

The FcεR family includes two types, designated FcεRI and FcεRII (also known as CD23). FcεRI is a high affinity receptor (binds IgE with an affinity of about 10¹⁰M⁻¹) found on mast, basophil and eosinophil cells that anchors monomeric IgE to the cell surface. The FcεRI possesses one alpha chain, one beta chain and the gamma chain homodimer discussed above. The FcεRII is a low affinity receptor expressed on mononuclear phagocytes, B lymphocytes, eosinophils and platelets. The FcεRII comprises a single polypeptide chain and does not include the gamma chain homodimer.

The FcγR family includes three types, designated FcγRI (also known as CD64), FγRII (also known as CD32) and FcγRIII (also known as CD 16). FcγRI is a high affinity receptor (binds IgGl with an affinity of 10⁸M⁻¹) found on mast, basophil, mononuclear, neutrophil, eosinophil, dendritic and phagocyte cells that anchors nomomeric IgG to the cell surface. The FcγRI includes one alpha chain and the gamma chain dimer shared by FcαRI and FcεRI.

The FcγRII is a low affinity receptor expressed on neutrophils, monocytes, eosinophils, platelets and B lymphocytes. The FcγRII includes one alpha chain, and does not include the gamma chain homodimer discussed above.

The FcγRIII is a low affinity (binds IgGl with an affinity of 5×10⁵M⁻¹) expressed on NK, eosinophil, macrophage, neutrophil and mast cells. It comprises one alpha chain and the gamma homodimer shared by FcαRI, FcεRI and FcγRI.

Skilled artisans will recognize that the subunit structure and binding properties of these various Fc receptors, as well as the cell types expressing them, are not completely characterized. The above discussion merely reflects the current state-of-the-art regarding these receptors (see, e.g., Immunobiology: The Immune System in Health & Disease, 5^{th} Edition, Janeway et al., Eds, 2001, ISBN 0-8153-3642-x, Figure 9.30 at pp. 371), and is not intended to be limiting with respect to the myriad receptor signaling cascades that can be regulated with the compounds described herein.

“Fc Receptor-Mediated Degranulation” or “Fc Receptor-Induced Degranulation” refers to degranulation that proceeds via an Fc receptor signal transduction cascade initiated by crosslinking of an Fc receptor.

“IgE-Induced Degranulation” or “FcεRI-Mediated Degranulation” refers to degranulation that proceeds via the IgE receptor signal transduction cascade initiated by crosslinking of FcεRl-bound IgE. The crosslinking may be induced by an IgE-specific allergen or other multivalent binding agent, such as an anti-IgE antibody. In mast and/or basophil cells, the FcεRI signaling cascade leading to degranulation may be broken into two stages: upstream and downstream. The upstream stage includes all of the processes that occur prior to calcium ion mobilization. The downstream stage includes calcium ion mobilization and all processes downstream thereof. Compounds that inhibit FcεRI-mediated degranulation may act at any point along the FcεRI-mediated signal transduction cascade. Compounds that selectively inhibit upstream FcεRI-mediated degranulation act to inhibit that portion of the FcεRI signaling cascade upstream of the point at which calcium ion mobilization is induced. In cell-based assays, compounds that selectively inhibit upstream FcεRI-mediated degranulation inhibit degranulation of cells such as mast or basophil cells that are activated or stimulated with an IgE-specific allergen or binding agent (such as an anti-IgE antibody) but do not appreciably inhibit degranulation of cells that are activated or stimulated with degranulating agents that bypass the FcεRI signaling pathway, such as, for example the calcium ionophores ionomycin and A23187.

“IgG-Induced Degranulation” or “FcγRI-Mediated Degranulation” refers to degranulation that proceeds via the FcγRI signal transduction cascade initiated by crosslinking of FcγRI-bound IgG. The crosslinking may be induced by an IgG-specific allergen or another multivalent binding agent, such as an anti-IgG or fragment antibody. Like the FcεRI signaling cascade, in mast and basophil cells the FcγRI signaling cascade also leads to degranulation which may be broken into the same two stages: upstream and downstream. Similar to FcεRI-mediated degranulation, compounds that selectively inhibit upstream FcγRI-mediated degranulation act upstream of the point at which calcium ion mobilization is induced. In cell-based assays, compounds that selectively inhibit upstream FcγRI-mediated degranulation inhibit degranulation of cells such as mast or basophil cells that are activated or stimulated with an IgG-specific allergen or binding agent (such as an anti-IgG antibody or fragment) but do not appreciably inhibit degranulation of cells that are activated or stimulated with degranulating agents that bypass the FcγRI signaling pathway, such as, for example the calcium ionophores ionomycin and A23187.

“Ionophore-Induced Degranulation” or “Ionophore-Mediated Degranulation” refers to degranulation of a cell, such as a mast or basophil cell, that occurs upon exposure to a calcium ionophore such as, for example, ionomycin or A23187.

“Syk Kinase” refers to the well-known 72 kDa non-receptor (cytoplasmic) spleen protein tyrosine kinase expressed in B-cells and other hematopoetic cells. Syk kinase includes two consensus Src-homology 2 (SH2) domains in tandem that bind to phosphorylated immunoreceptor tyrosine-based activation motifs (“ITAMs”), a “linker” domain and a catalytic domain (for a review of the structure and function of Syk kinase see Sada et al., 2001, J. Biochem. (Tokyo) 130:177-186); see also Turner et al., 2000, Immunology Today 21:148-154). Syk kinase has been extensively studied as an effector of B-cell receptor (BCR) signaling (Turner et al., 2000, supra). Syk kinase is also critical for tyrosine phosphorylation of multiple proteins which regulate important pathways leading from immunoreceptors, such as Ca²⁺ mobilization and mitogen-activated protein kinase (MAPK) cascades and degranulation. Syk kinase also plays a critical role in integrin signaling in neutrophils (see, e.g., Mocsai et al. 2002, Immunity 16:547-558).

As used herein, Syk kinase includes kinases from any species of animal, including but not limited to, homosapiens, simian, bovine, porcine, rodent, etc., recognized as belonging to the Syk family. Specifically included are isoforms, splice variants, allelic variants, mutants, both naturally occurring and man-made. The amino acid sequences of such Syk kinases are well known and available from GENBANK. Specific examples of mRNAs encoding different isoforms of human Syk kinase can be found at GENBANK accession no. gi|21361552|ref|NM_{—}003177.2|, gi|496899|emb|Z29630.1|HSSYKPTK[496899] and gi|15030258|gb|BC011399.1|BC011399[15030258], which are incorporated herein by reference.

Skilled artisans will appreciate that tyrosine kinases belonging to other families may have active sites or binding pockets that are similar in three-dimensional structure to that of Syk. As a consequence of this structural similarity, such kinases, referred to herein as “Syk mimics,” are expected to catalyze phosphorylation of substrates phosphorylated by Syk. Thus, it will be appreciated that such Syk mimics, signal transduction cascades in which such Syk mimics play a role, and biological responses effected by such Syk mimics and Syk mimic-dependent signaling cascades may be regulated, and in particular inhibited, with many of the compounds described herein.

“Syk-Dependent Signaling Cascade” refers to a signal transduction cascade in which Syk kinase plays a role. Non-limiting examples of such Syk-dependent signaling cascades include the FcαRI, FcεRI, FcγRI, FcγRIII, BCR and integrin signaling cascades.

“Autoimmune Disease” refers to those diseases which are commonly associated with the nonanaphylactic hypersensitivity reactions (Type II, Type III and/or Type IV hypersensitivity reactions) that generally result as a consequence of the subject's own humoral and/or cell-mediated immune response to one or more immunogenic substances of endogenous and/or exogenous origin. Such autoimmune diseases are distinguished from diseases associated with the anaphylactic (Type I or IgE-mediated) hypersensitivity reactions.

### Methods of Synthesis

The compounds described herein, as well as intermediates thereof, may be synthesized via a variety of different synthetic routes using commercially available starting materials and/or starting materials prepared by conventional synthetic methods. Suitable exemplary methods that may be routinely used and/or adapted to synthesize active deuterated 2,4-pyrimidinediamine compounds can be found in international patent application no. PCT/US03/03022 filed Jan. 31, 2003 (WO 03/063794), international patent application no. PCT/US07/85313 filed Nov. 20, 2007 (WO 2008/064274), and international patent application no. PCT/US06/01945 filed Jan. 19, 2006 (WO 2006/078846). Each of these prior publications is incorporated herein by reference. Representative synthetic procedures for the preparation of compounds described herein are outlined below in Schemes 1-5, below:

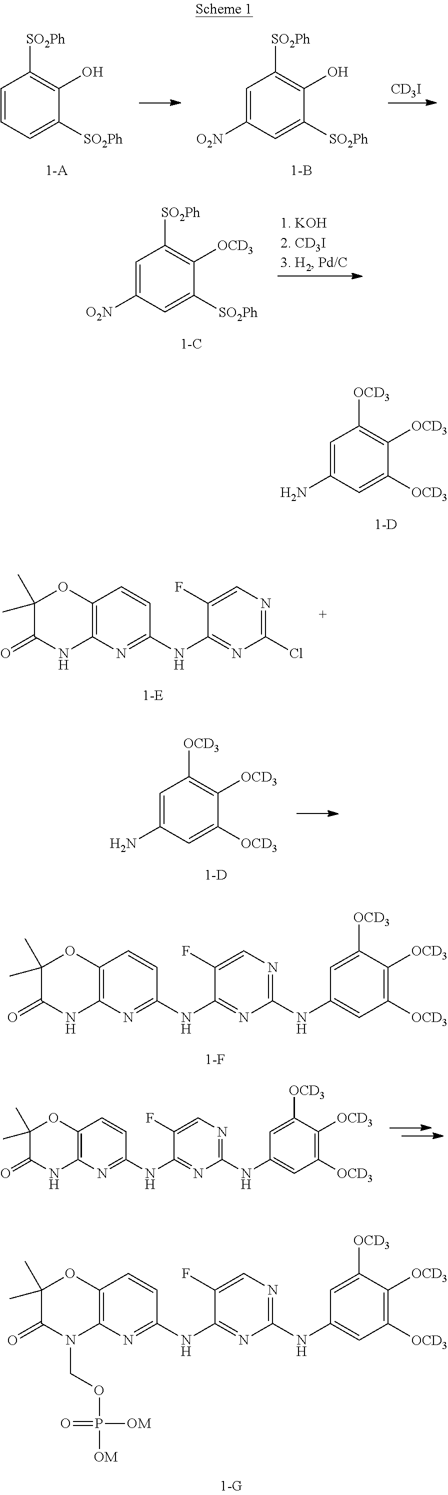

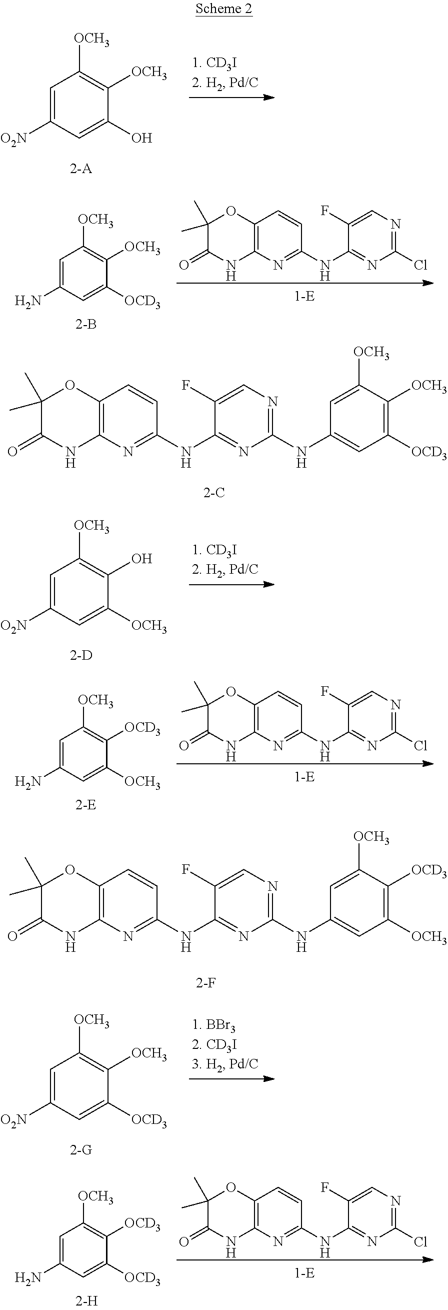

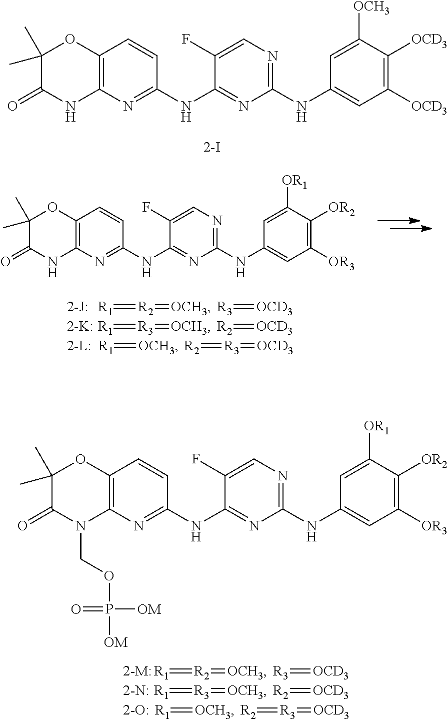

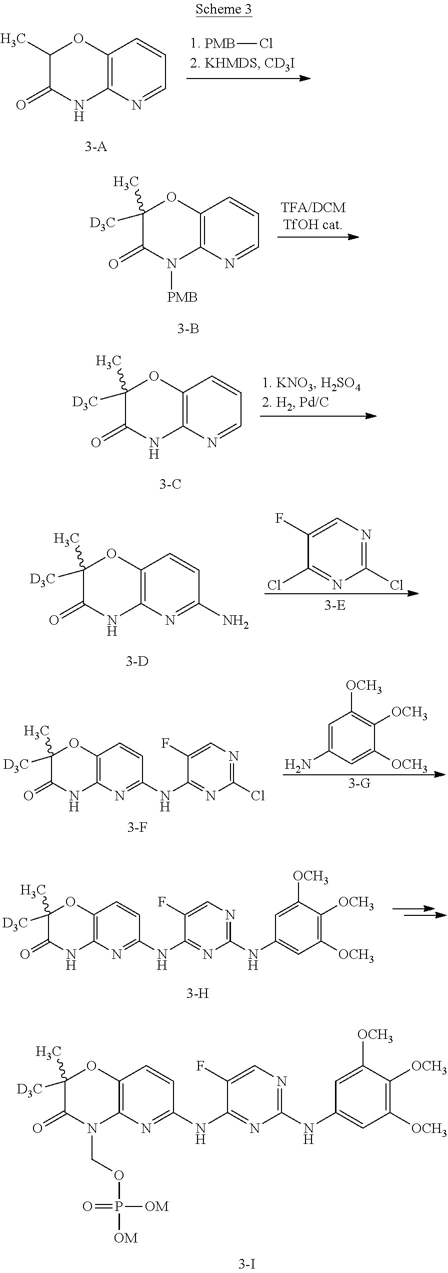

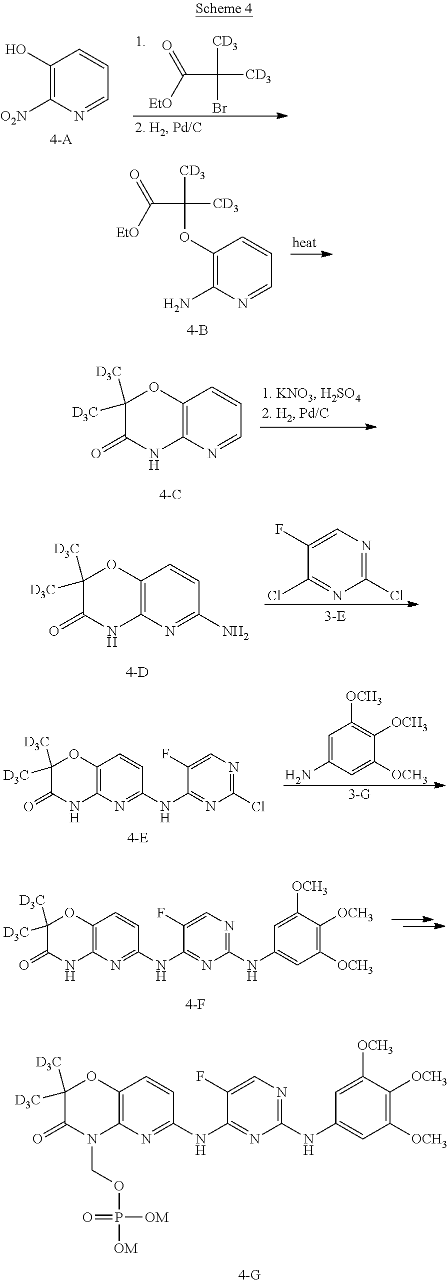

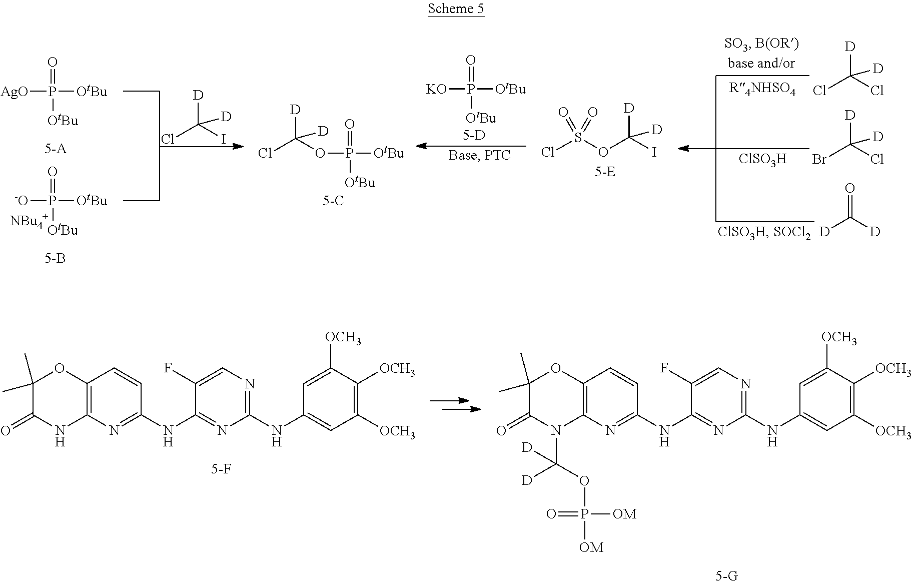

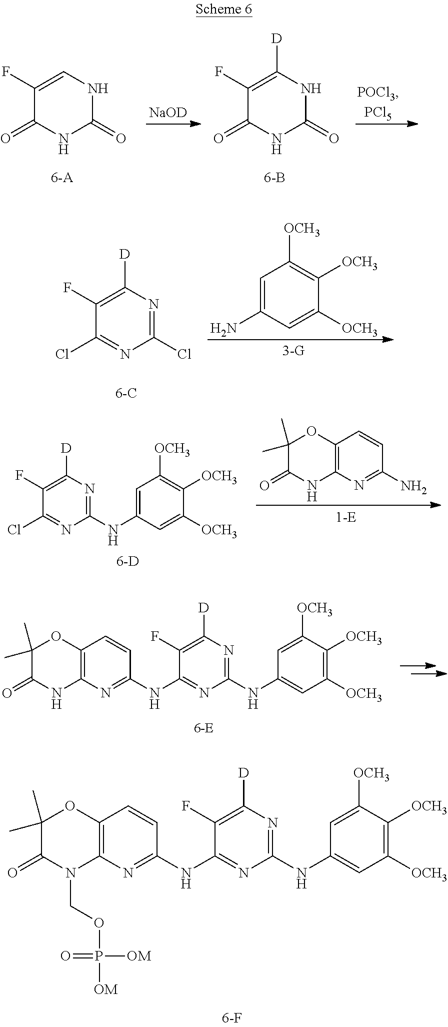

Those having skill in the art will recognize that the starting materials and reaction conditions may be varied, the sequence of the reactions altered, and additional steps employed to produce compounds encompassed by the present disclosure. Skilled artisans will recognize that in some instances, during the synthesis of deuterated 2,4-pyrimidinediamine compounds and prodrugs thereof, the starting materials may include functional groups that require protection during synthesis. The exact identity of any protecting group(s) used will depend upon the identity of the functional group being protected, and will be apparent to these of skill in the art. In general, the need for such protecting groups as well as the conditions necessary to attach and remove such groups will be apparent to those skilled in the art of organic synthesis.

### Pharmaceutical Compositions

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known.

For buccal administration, the compositions may take the form of tablets or lozenges formulated in conventional manner.

For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. The compound(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an allergy provides therapeutic benefit not only when the underlying allergic response is eradicated or ameliorated, but also when the patient reports a decrease in the severity or duration of the symptoms associated with the allergy following exposure to the allergen. As another example, therapeutic benefit in the context of asthma includes an improvement in respiration following the onset of an asthmatic attack, or a reduction in the frequency or severity of asthmatic episodes. Therapeutic benefit in the context of rheumatoid arthritis also includes the ACR20, or ACR50 or ACR70, as previously described. Therapeutic benefit also generally includes halting or slowing the progression of the disease, regardless of whether improvement is realized.

For prophylactic administration, the compound(s) may be administered to a patient at risk of developing one of the previously described diseases. For example, if it is unknown whether a patient is allergic to a particular drug, the compound(s) may be administered prior to administration of the drug to avoid or ameliorate an allergic response to the drug. Alternatively, prophylactic administration may be applied to avoid the onset of symptoms in a patient diagnosed with the underlying disorder. For example, the compound(s) may be administered to an allergy sufferer prior to expected exposure to the allergen. Compound(s) may also be administered prophylactically to healthy individuals who are repeatedly exposed to agents known to one of the above-described maladies to prevent the onset of the disorder. For example, compound(s) may be administered to a healthy individual who is repeatedly exposed to an allergen known to induce allergies, such as latex, in an effort to prevent the individual from developing an allergy. Alternatively, compound(s) may be administered to a patient suffering from asthma prior to partaking in activities which trigger asthma attacks to lessen the severity of, or avoid altogether, an asthmatic episode.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active metabolite compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective local dosages without undue experimentation.

### EXAMPLES

The preparation of the compounds of the disclosure is illustrated further by the following examples, which are provided for illustrative purposes and are not intended to be construed as limiting the disclosure in scope or spirit to the specific procedures and compounds described in them.

### Example 2-C

### N4-[2,2-Dimethyl-3-oxo-pyrid[1,4]oxazin-6-yl]-5-fluoro-N2-(3,4-dimethoxyphenyl-5-trideuteromethoxy)-2,4-pyrimidinediamine (Compound 2-C)

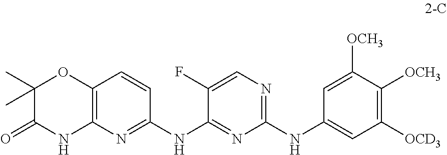

A mixture of 330 mg of 2-chloro-5-fluoro-N-4-[2,2-dimethyl-3-oxo-pyrid[1,4]oxazin-6-yl]-4-pyrimidineamine and 380 mg of 3,4-dimethoxy-5-trideuteromethoxyaniline in 3 mL EtOH with 5 drops of 4 N HCl solution in 1,4-dioxane was heated in the microwave at 175° C. for 3600 seconds. The precipitate formed was collected by suction filtration, dried, and neutralized with dilute potassium carbonate solution. The solid was collected by suction filtration and dried to yield 275 mg of the desired product N4-[2,2-dimethyl-3-oxo-pyrid[1,4]oxazin-6-yl]-5-fluoro-N2-(3,4-dimethoxyphenyl-5-trideuteromethoxy)-2,4-pyrimidinediamine. ¹H NMR (DMSO-d6): δ 11.06 (s, 1H), 9.15 (s, 1H), 9.10 (s, 1H), 8.12 (d, 1H, J=3.6 Hz), 7.66 (d, 1H, J=8.1 Hz), 7.31 (d, 1H, J=8.1 Hz), 7.02 (s, 2H), 3.64 (s, 3H), 3.58 (s, 3H), 1.41 (s, 6H); purity 99%; MS (m/e): 474 (MH⁺).

### Example 2-F

### N4-[2,2-Dimethyl-3-oxo-pyrid[1,4]oxazin-6-yl]-5-fluoro-N2-(3,5-dimethoxyphenyl-4-trideuteromethoxy)-2,4-pyrimidinediamine (Compound 2-F)

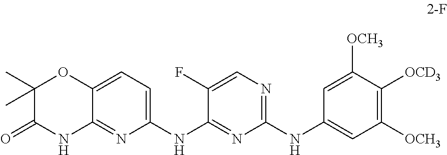

N4-[2,2-Dimethyl-3-oxo-pyrid[1,4]oxazin-6-yl]-5-fluoro-N2-(3,5-dimethoxyphenyl-4-trideuteromethoxy)-2,4-pyrimidinediamine was prepared in a manner analogous to that described in Example 2-C. ¹H NMR (DMSO-d6): δ 11.06 (s, 1 Hz), 9.15 (s, 1H), 9.10 (s, 1H), 8.12 (d, 1H, J=3.6 Hz), 7.66 (d, 1H, J=8.1 Hz), 7.31 (d, 1H, J=8.1 Hz), 7.02 (s, 2H), 3.60 (s, 6H), 1.41 (s, 6H); purity 99%; MS (m/e): 474 (MH⁺).

### Example 2-I

### N4-[2,2-Dimethyl-3-oxo-pyrid[1,4]oxazin-6-yl]-5-fluoro-N2-(3-methoxy-4,5-bis-trideuteromethoxyphenyl)-2,4-pyrimidinediamine (Compound 2-I)

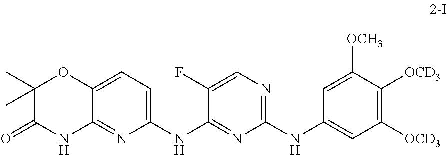

N4-[2,2-Dimethyl-3-oxo-pyrid[1,4]oxazin-6-yl]-5-fluoro-N2-(3-methoxy-4,5-bis-trideuteromethoxyphenyl)-2,4-pyrimidinediamine was prepared in a manner analogous to that described in Example 2-I. ¹H NMR (DMSO-d6): ¹H NMR (DMSO-d6): 11.06 (s, 1H), 9.15 (s, 1H), 9.10 (s, 1H), 8.12 (d, 1H, J=3.6 Hz), 7.66 (d, 1H, J=8.1 Hz), 7.31 (d, 1H, J=8.1 Hz), 7.02 (s, 2H), 3.61 (s, 3H), 1.41 (s, 6H); purity 90%; MS (m/e): 477 (MH⁺).

### Example 5-C

### Di-tert-butyl chlorodideuteromethyl phosphate (Compound 5-C)

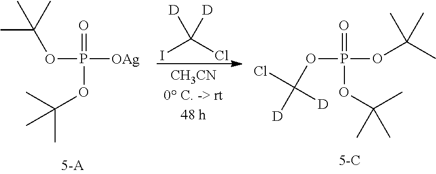

CD₂ICl (1.0 g, 5.60 mmol) in dry acetonitrile (5 mL) was added to a stirring heterogeneous solution of silver di-tert-butylphosphate (5-A; 1.25 g, 3.94 mmol) in acetonitrile (100 mL) at 0° C. for 20 min. External cooling was removed after 2 h, allowed to warm to room temperature and continued the stirring of the heterogeneous reaction mixture for 48 h. The reaction mixture was filtered and the filtrate concentrated. The semi precipitous crude was suspended in EtOAc (50 mL) and filtered. The filtrate was concentrated and dried under vacuum. The concentrate was analyzed to provide the desired product 5-C which was used without further purification in the next step. ¹H NMR (CDCl₃): δ 1.46 (s, 6H); ³¹P NMR (CDCl₃): δ −10.81.

### Example 5-G

### N4-(2,2-Dimethyl-4-[(di-tert-butyl phosphonoxy)dideuteromethyl]-3-oxo-5-pyrido[1,4]oxazin-6-yl)-5-fluoro-N2-(3,4,5-trimethoxyphenyl)-2,4-pyrimidinediamine (Compound 2)

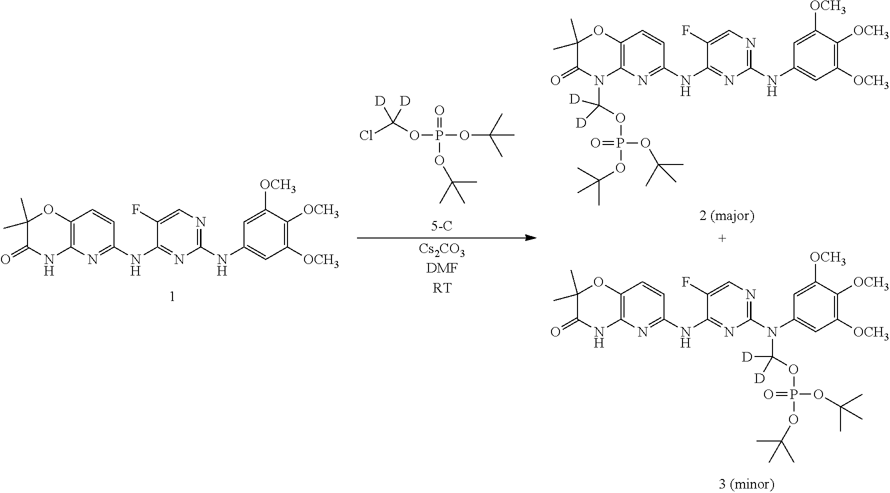

N4-(2,2-dimethyl-3-oxo-4H-5-pyrido[1,4]oxazin-6-yl)-5-fluoro-N2-(3,4,5-trimethoxyphenyl)-2,4-pyrimidinediamine (1, 0.371 g, 0.789 mmol), Cs₂CO₃ (0.30 g, 0.92 mmol) and di-tent-butyl chlorodideuteromethyl phosphate (0.267 g, 1.02 mmol) in DMF (5 mL) was stirred at room temperature under nitrogen atmosphere. Progress of the reaction was monitored by in process LC/MS. Crude reaction mixture displayed two product peaks (ratio 1:5) with close retention times displaying M⁺+H 695 (minor) and 695 (major) besides starting material (1). Initial yellow reaction mixture turned to olive green as the reaction progressed. Reaction mixture was added to stirring water (10 ml), filtered the precipitate, washed with water and dried to provide off-white crude product. The crude was purified by column chromatography to obtain the major product N4-(2,2-dimethyl-4-[(di-tert-butyl phosphonoxy)dideuteromethyl]-3-oxo-5-pyrido[1,4]oxazin-6-yl)-5-fluoro-N2-(3,4,5-trimethoxyphenyl)-2,4-pyrimidinediamine (2) (150 mg, 90% pure). ¹H NMR (CDCl₃): δ 7.98 (d, 1H, J=8.5 Hz), 7.97 (d, 1H, J=4.1 Hz), 7.74 (s, 1H), 7.40 (s, 1H), 7.19 (d, 1H, J=8.5 Hz), 6.76 (s, 2H), 3.80 (s, 3H), 3.77 (s, 6H), 1.48 (s, 6H), 1.44 (s, 18H). ³¹P NMR (CDCl₃): −11.15; LCMS: MS (m/e): 695 (MH⁺).

### N4-(2,2-Dimethyl-4-[(dihydrogen phosphonoxy)dideuteromethyl]-3-oxo-5-pyrido[1,4]oxazin-6-yl)-5-fluoro-N2-(3,4,5-trimethoxyphenyl)-2,4-pyrimidinediamine (Compound 5-G)

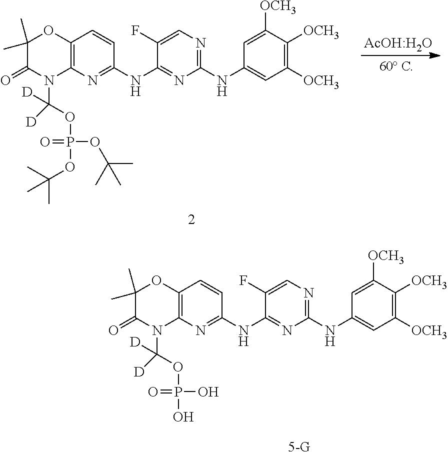

N4-(2,2-dimethyl-4-[(di-tert-butyl phosphonoxy)dideuteromethyl]-3-oxo-5-pyrido[1,4]oxazin-6-yl)-5-fluoro-N2-(3,4,5-trimethoxyphenyl)-2,4-pyrimidinediamine (1; 120 mg) dissolved in AcOH:H₂O (2 mL, 4:1) was heated at 60° C. (oil bath temp). The progress of the reaction was monitored by in process LC/MS. The reaction mixture transformed to faint tan white solid after 1 h of heating. The heating was stopped, cooled the reaction mixture to room temperature, diluted with acetone (10 mL) and stirred for 20 min. The suspension was filtered and the collected solid was washed with acetone (10 mL) and suction dried to provide N4-(2,2-dimethyl-4-[(dihydrogen phosphonoxy)dideuteromethyl]-3-oxo-5-pyrido[1,4]oxazin-6-yl)-5-fluoro-N2-(3,4,5-trimethoxyphenyl)-2,4-pyrimidinediamine (5-G) (100 mg). ¹H NMR (DMSO-d₆): δ 9.22 (s, 2H), 8.17 (d, 1H, J=3.2 Hz), 7.95 (d, 1H, J=8.5 Hz), 7.39 (d, 1H, J=8.5 Hz), 7.05 (s, 2H), 3.67 (s, 6H), 3.59 (s, 3H), 1.44 (s, 6H). ³¹P NMR (CDCl₃): −2.13; LCMS: MS (m/e): 583 (MH⁺).

### Example 2-O

### N4-(2,2-Dimethyl-4-[(dihydrogen phosphonoxy)methyl]-3-oxo-5-pyrido[1,4]oxazin-6-yl)-5-fluoro-N2-(3,4-dimethoxy-5-trideuteromethoxyphenyl)-2,4-pyrimidinediamine Di Sodium Salt (Compound 2-O)

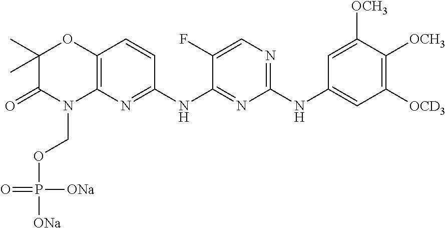

N4-(2,2-Dimethyl-4-[(dihydrogen phosphonoxy)methyl]-3-oxo-5-pyrido[1,4]oxazin-6-yl)-5-fluoro-N2-(3,4-dimethoxy-5-trideuteromethoxyphenyl)-2,4-pyrimidinediamine Di Sodium Salt was prepared using standard methodologies. ¹H NMR (D₂O): δ 7.77 (d, 1H, J=3.5 Hz), 7.59 (d, 1H, J=8.8 Hz), 7.01 (d, 1H, J=8.5 Hz), 6.61 (s, 2H), 5.56 (d, 2H, J=2.7 Hz), 3.60 (s, 3H), 3.56 (s, 3H), 1.37 (s, 6H). ³¹P NMR (D₂O): −2.57. LCMS: MS (m/e): 584 (MH⁺-2Na+2H).

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of formula (I′): 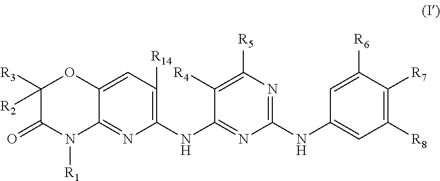
or a pharmaceutically acceptable salt thereof, wherein:
R₁ is selected from hydrogen C₁-C₆ alkyl, and R₉;
R₂ and R₃ are independently selected from hydrogen, halogen, halo(C₁-C₆)alkyl, and C₁-C₆ alkyl, or
R₂ and R₃ together form an oxo group;
R₄ is selected from hydrogen, halogen, cyano, nitro, C₁-C₆ alkyl, and halo(C₁-C₆)alkyl;
R₅ is hydrogen; and
R₆, R₇, and R₈ are independently selected from hydrogen, halogen, cyano, nitro, C₁-C₆ alkyl, C₂-C₆ alkenyl, C₂-C₆ alkynyl, phenyl, benzyl, —OR₁₀, —C(O)R₁₀, —C(O)OR₁₀, —NR₁₀R₁₀, —S(O)₂NR₁₀R₁₀, —C(O)NR₁₀R₁₀, —N(R)S(O)₂R₁₀ and —NC(O)OR₁₀, where each R₁₀ is independently hydrogen or C₁-C₆ alkyl; and
R₉ is —(CR₁₁R₁₁)ₖ—O—P(O)(OR₁₂)₂ or 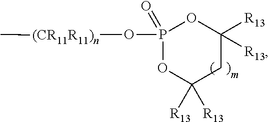
in which k is 1, 2 or 3; m is 0 or 1; n is 1, 2 or 3; each R₁₁ is independently hydrogen or C₁-C₆ alkyl; each R₁₂ is independently hydrogen or C₁-C₆ alkyl, and each R₁₃ is independently hydrogen, or C₁-C₆ alkyl; and
R₁₄ is hydrogen;
provided that at least one hydrogen of the compound is enriched in deuterium.

2. A compound according to claim 1 of formula (I′): 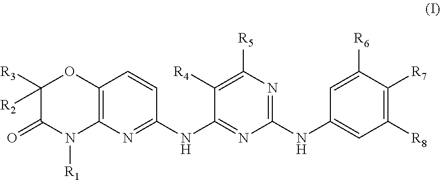
or a pharmaceutically acceptable salt thereof, wherein:
R₁ is selected from hydrogen, deuterium, C₁-C₆ alkyl optionally substituted with deuterium, and R₉;
R₂ and R₃ are independently selected from hydrogen, deuterium, halogen, halo(C₁-C₆)alkyl, and C₁-C₆ alkyl optionally substituted with deuterium, or
R₂ and R₃ together form an oxo group;
R₄ is selected from hydrogen, deuterium, halogen, cyano, nitro, C₁-C₆ alkyl, and halo(C₁-C₆)alkyl;
R₅ is hydrogen or deuterium; and
R₆, R₇, and R₈ are independently selected from hydrogen, deuterium, halogen, cyano, nitro, C₁-C₆ alkyl optionally substituted with deuterium, C₂-C₆ alkenyl optionally substituted with deuterium, C₂-C₆ alkynyl optionally substituted with deuterium, phenyl, benzyl, —OR₁₀, —C(O)R₁₀, —C(O)OR₁₀, —NR₁₀R₁₀, —S(O)₂NR₁₀R₁₀, —C(O)NR₁₀R₁₀, —N(R)S(O)₂R₁₀ and —NC(O)OR₁₀,
where each R₁₀ is independently selected from hydrogen, deuterium, and C₁-C₆ alkyl optionally substituted with deuterium; and
R₉ is selected from —(CR₁₁R₁₁)ₖ—O—P(O)(OR₁₂)₂ and 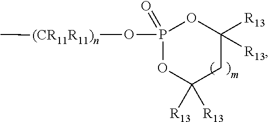
in which k is 1, 2 or 3; m is 0 or 1; n is 1, 2 or 3; each R₁₁ is independently selected from hydrogen, deuterium, and C₁-C₆ alkyl optionally substituted with deuterium; each R₁₂ is independently selected from hydrogen, deuterium, and C₁-C₆ alkyl optionally substituted with deuterium and each R₁₃ is independently selected from hydrogen, deuterium, and C₁-C₆ alkyl optionally substituted with deuterium;
provided that at least one deuterium is present.

3. (canceled)

4. (canceled)

5. The compound according to claim 1 wherein one or more of the positions substituted with deuterium has an enrichment factor of at least 6600.

6. The compound according to claim 2, wherein R₁ is selected from hydrogen, deuterium, and R₉.

7. (canceled)

8. (canceled)

9. (canceled)

10. The compound according to claim 9, wherein R₁ is —CD₂-O—P(O)(OH)₂.

11. The compound according to claim 1, wherein R₂ and R₃ are independently selected from hydrogen, deuterium, halogen, halo(C₁-C₆)alkyl, and C₁-C₆ alkyl optionally substituted with deuterium.

12. The compound according to claim 11, wherein R₂ and R₃ are independently selected from C₁-C₆ alkyl optionally substituted with deuterium.

13. The compound according to claim 12, wherein R₂ and R₃ are independently selected from —CH₃, —CH₂D, —CHD₂ and —CD₃.

14. (canceled)

15. (canceled)

16. (canceled)

17. The compound according to claim 2, wherein R₄ is halogen.

18. The compound according to claim 2, wherein R₅ is hydrogen.

19. The compound according to claim 2, wherein R₅ is deuterium.

20. The compound according to claim 2, wherein R₆, R₇, and R₈ are independently selected from —OR₁₀, in which each R₁₀ is independently selected from hydrogen, deuterium, and C₁-C₆ alkyl optionally substituted with deuterium.

21. The compound according to claim 20, wherein R₆, R₇, and R₈ are independently selected from —OR₁₀, in which each R₁₀ is C₁-C₆ alkyl optionally substituted with deuterium.

22. The compound according to claim 21, wherein R₆, R₇, and R₈ are independently selected from —OCH₃, —OCH₂D, —OCHD₂ and —OCD₃.

23. The compound according to claim 22, wherein at least one of R₆, R₇, or R₈ is —OCD₃.

24. The compound according to claim 2, wherein at least two of R₁, R₂, R₃, R₅, R₆, R₇, and R₈ contain deuterium.

25. The compound according to claim 1, wherein:
R₁ is hydrogen or —CH₂—O—P(O)(OH)₂;
R₂ and R₃ are independently C₁-C₆ alkyl;
R₄ is halogen;
R₅ is hydrogen; and
R₆, R₇, and R₈ are independently selected from —OR₁₀, in which each R₁₀ is C₁-C₆ alkyl optionally substituted with deuterium, and
at least one of R₆, R₇, or R₈ contains deuterium.

26. The compound according to claim 1, wherein:
R₁ is hydrogen or —CH₂—O—P(O)(OH)₂;
R₂ and R₃ are independently C₁-C₆ alkyl optionally substituted with deuterium, where at least one of R₂ or R₃ contains deuterium;
R₄ is halogen;
R₅ is hydrogen; and
R₆, R₇, and R₈ are independently selected from —O(C₁-C₆ alkyl).

27. The compound according to claim 1, wherein:
R₁ is —CD₂-O—P(O)(OH)₂;
R₂ and R₃ are independently C₁-C₆ alkyl;
R₄ is halogen;
R₅ is hydrogen; and
R₆, R₇, and R₈ are independently selected from —O(C₁-C₆ alkyl).

28. The compound according to claim 1, wherein:
R₁ is hydrogen or —CH₂—O—P(O)(OH)₂;
R₂ and R₃ are independently C₁-C₆ alkyl;
R₄ is halogen;
R₅ is deuterium; and
R₆, R₇, and R₈ are independently selected from —O(C₁-C₆ alkyl).

29. (canceled)

30. (canceled)

31. The compound according to claim 24 wherein one or more of the positions substituted with deuterium has an enrichment factor of at least 6600.

32. The pharmaceutically acceptable salt of a compound according to claim 2.

33. The pharmaceutically acceptable salt of claim 32, wherein in the compound R₁ is —(CR₁₁R₁₁)ₖ—O—P(O)(OR₁₂)₂ in which at least one R₁₂ is hydrogen or deuterium.

34. The salt according to claim 33, which is a mono- or di-sodium salt, or mono- or di-potassium salt.

35. The salt according to claim 33, which is a di-sodium or di-potassium salt.

36. (canceled)

37. (canceled)

38. The compound or a salt according to claim 1, which is selected from the group consisting of: 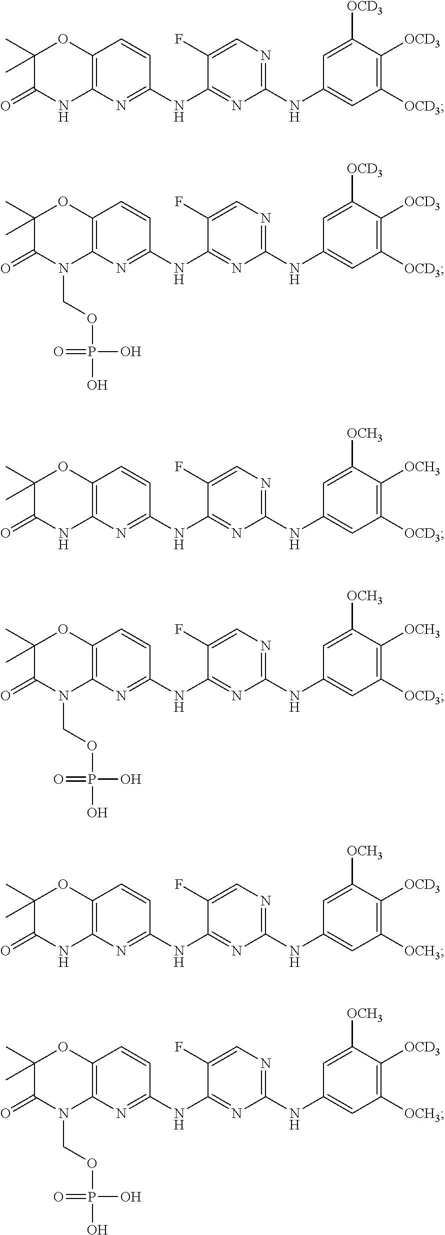 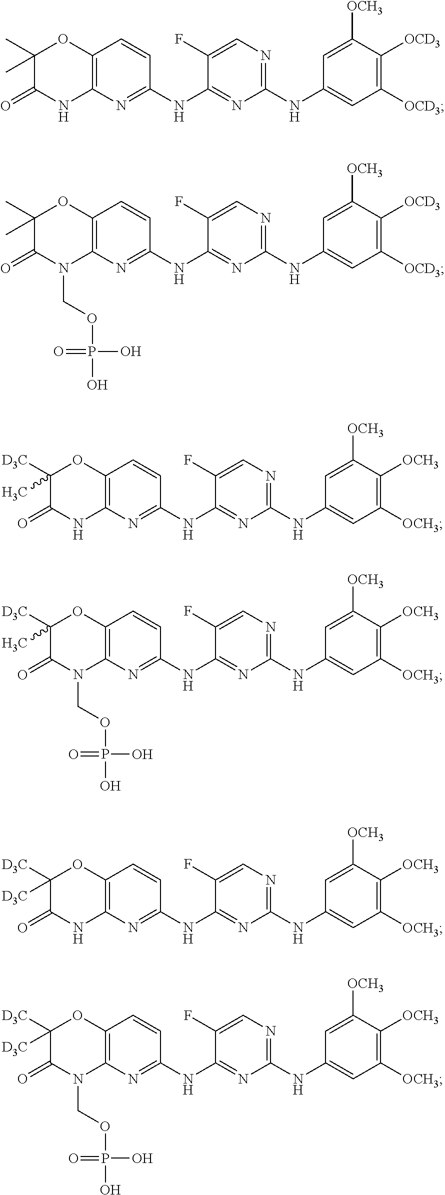 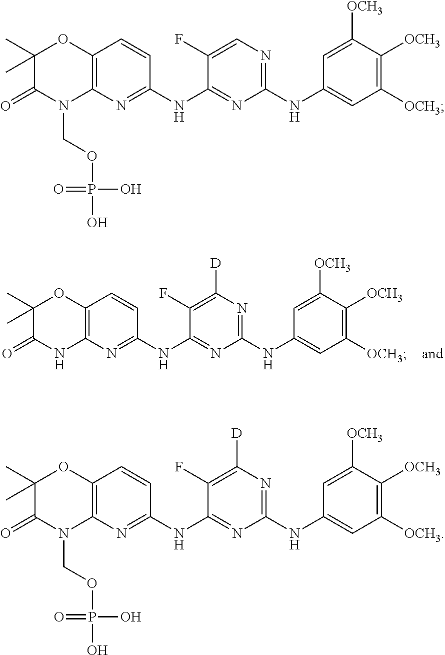
and pharmaceutically-acceptable salts thereof.

39. (canceled)

40. (canceled)

40. The compound according to claim 38 wherein one or more of the positions substituted with deuterium has an enrichment factor of at least 6600.

42. A compound which is di-tent-butyl chlorodideuteromethyl phosphate.

43. A method of preparation of di-tent-butyl chlorodideuteromethyl phosphate, comprising reacting a di-tent-butyl phosphate salt with CD₂ICl.

44. A method of preparation of the compound having the formula:
X₁—(CR₁₁R₁₁)ₖ—O—P(O)(OH)₂
wherein k is 1, 2 or 3; X₁ is halogen; and each R₁₁ is independently selected from hydrogen, deuterium, and C₁-C₆ alkyl optionally substituted with deuterium;
the method comprising reacting a di-tert-butylphosphate salt with X₁—(CR₁₁R₁₁)ₖ—X₂, in which X₂ is halogen.

45. The method according to claim 43 wherein the di-tert-butylphosphate salt is silver di-tent-butyl phosphate or tetrabutylammonium di-tent-butyl phosphate.

46. A pharmaceutical composition comprising a compound or salt according to claim 2 and an acceptable carrier, excipient and/or diluent.

47. A method of inhibiting cell degranulation in a subject, comprising administering to the subject a pharmaceutically effective amount of a compound or salt according to claim 2 effective to inhibit degranulation.

48. A method of treating or preventing a disease in a subject, where the disease is selected from an allergic disease, low grade scarring, a disease associated with tissue destruction, a disease associated with tissue inflammation, inflammation and scarring, comprising administering to the subject a pharmaceutically effective amount of a compound or salt according to claim 2.

49. The method according to claim 48 in which the disease is rheumatoid arthritis.

50. A method of inhibiting an activity of a Syk kinase in a subject, comprising administering to the subject a pharmaceutically effective amount of a compound or salt according to claim 2 effective to inhibit the Syk kinase activity.

51. (canceled)

52. (canceled)

53. A method of treating or preventing an autoimmune disease in a subject, and/or one or more symptoms associated therewith, comprising administering to the subject a pharmaceutically effective amount of a compound or salt according to claim 2 effective to treat or prevent the autoimmune disease.

54. The method according to claim 53 in which the autoimmune disease is selected from autoimmune diseases that are frequently designated as single organ or single cell-type autoimmune disorders and autoimmune disease that are frequently designated as involving systemic autoimmune disorder.

55. The method according to claim 53 in which the autoimmune disease is selected from Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, chronic aggressive hepatitis, ulcerative colitis, membranous glomerulopathy, systemic lupus erythematosis, rheumatoid arthritis, Sjogren's syndrome, Reiter's syndrome, polymyositis-dermatomyositis, systemic sclerosis, polyarteritis nodosa, multiple sclerosis and bullous pemphigoid.

56. A method of treating rheumatoid arthritis in a subject, comprising administering to a subject suffering from rheumatoid arthritis a pharmaceutically effective amount of a compound or salt according to claim 2.

57. The method according to claim 56 in which the amount of the compound administered is effective to achieve a serum concentration of the corresponding drug that is at or above the IC₅₀ of Syk inhibition of the drug, as measured in an in vitro assay.
